(19) United States
(12) Patent Application Publication
(10) Pub. No.: US 2024/0207354 A1
(43) Pub. Date: Jun. 27, 2024

(54) ETELCALCETIDE FORMULATIONS

(71) Applicant: AMGEN INC., Thousand Oaks, CA (US)

(72) Inventors: David Lai, Port Hueneme, CA (US); Iman Mirzaee Kakhki, Wayland, MA (US)

(21) Appl. No.: 18/288,410

(22) PCT Filed: May 3, 2022
(86) PCT No.: PCT/US22/27451

§ 371 Date: Oct. 26, 2023

Related U.S. Application Data
(60) Provisional application No. 63/184,924, filed on May 6, 2021.

(51) Int. Cl. A61K 38/08; A61K 9/08; A61K 47/26

(52) U.S. Cl. CPC A61K 38/08; A61K 9/08; A61K 47/26

(57) ABSTRACT

Disclosed herein are pharmaceutical formulations comprising etelcalcetide or salt thereof and a surfactant in aqueous solution, wherein the formulation has a pH of 2.0 to 5.0.

### CROSS-REFERENCE TO RELATED APPLICATIONS

This application claims the benefit of priority to U.S. Provisional Patent Application No. 63/184,924, filed May 6, 2021, which is hereby incorporated by reference in its entirety.

### BACKGROUND

In any pharmaceutical packaging process, error rates must be kept very low to prevent defective or incorrect medicine from reaching the consumer. Stable liquid formulations of etelcalcetide have been described previously (International Publication No. WO 2014/210489). The present disclosure reports that high surface tension and low viscosity compositions (such as etelcalcetide liquid formulations) can form droplets on the interior surface of the vial. Droplets on the interior surface of a vial create shadows on the vial that the automated vial visual inspection system identifies as being cracks, resulting in the false rejection of such vials. There remains a need in the art for liquid etelcalcetide formulations that do not promote formation of droplets on the internal surface of vials, thus lowering the false rejection rate during the packaging process.

### SUMMARY

The present disclosure provides pharmaceutical formulations comprising etelcalcetide and a surfactant in aqueous solution, wherein the formulation has a pH of 2 to 5. In some embodiments, the formulations further comprise a tonicity modifier. Exemplary tonicity modifiers include, but are not limited to, sodium chloride, mannitol, sucrose, dextrose, sorbitol, potassium chloride, or mixtures thereof.

In some embodiments, the etelcalcetide is present in the formulation at a concentration of about 1 mg/ml to 20 mg/ml or about 1 mg/L, or about 2.5 mg/mL, or about 5 mg/ml or about 10 mg/mL.

In some embodiments, the formulations have a pH of 3 to 4. In some embodiments, the pH is maintained by a pharmaceutically acceptable buffer. Exemplary buffers include, but are not limited to, succinate, citrate, malate, edentate, histidine, acetate, adipate, aconitate, ascorbate, benzoate, carbonate, bicarbonate, maleate, glutamate, lactate, phosphate, and tartrate or a mixture thereof. In some embodiments, the buffer is succinate.

The disclosure also provides pharmaceutical formulations comprising etelcalcetide hydrochloride and a surfactant in aqueous solution, wherein the formulation has a pH of 2.0 to 5.0. In some embodiments, the etelcalcetide hydrochloride is present in the formulation at a concentration of 1 mg/ml to 15 mg/mL.

In some embodiments, the surfactant in the formulations disclosed herein comprises polysorbate 20, polysorbate 40, polysorbate 60, polysorbate 80, poloxamer 188, poloxamer 407, triton X-100, polyoxyethylene, PEG 3350, PEG 4000, sodium dodecyl sulfate (SDS), polyvinyl alcohol (PVA), phosphatidylcholine (PC), Pluronic F-127, or a combination thereof. In some embodiments, the surfactant is present in the formulation at a concentration of 0.005% (w/v) to 0.05% (w/v). In some embodiments, the surfactant is polysorbate 20 or polysorbate 80. In some embodiments, the surfactant is polysorbate 20.

The disclosure also provides pharmaceutical formulations comprising 1 mg/ml to 20 mg/ml of etelcalcetide hydrochloride in aqueous solution, a succinate buffer that maintains the formulation at a pH of 3 to 4, a surfactant, and a concentration of sodium chloride wherein the formulation is approximately isotonic.

The disclosure also provides pharmaceutical formulations comprising etelcalcetide or salt thereof at a concentration of 1 mg/ml to 20 mg/mL in aqueous solution; a succinate buffer at a concentration that maintains the formulation at a pH of about 3 to 4; a surfactant, and sodium chloride at a concentration such that the formulation is approximately isotonic.

It should be understood that while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” the feature. It is to be noted that the term “a” or “an” refers to one or more, for example, “an immunoglobulin molecule,” is understood to represent one or more immunoglobulin molecules. As such, the terms “a” (or “an”), “one or more,” and “at least one” can be used interchangeably herein.

When the term “about” is used, it means the recited number plus or minus 5%, 10%, 15% or more of that recited number. The actual variation intended is determinable from the context.

In any of the ranges described herein, the endpoints of the range are included in the range. However, the description also contemplates the same ranges in which the lower and/or the higher endpoint is excluded. Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

All references cited herein are hereby incorporated by reference in their entireties.

### DETAILED DESCRIPTION

Stable liquid formulations of etelcalcetide have been described previously (International Publication No. WO 2014/210489). As described in Example 1, the present disclosure reports that liquid etelcalcetide formulations have high surface tension and low viscosity (such as etelcalcetide liquid formulations), a combination that increases the propensity of droplet formation on the interior surface of a vial during the packaging process. Such interior droplets cast shadows on the vial that the automated vial visual inspection system identifies as being cracks, resulting in the false rejection of such vials. The present disclosure is based on the discovery that the inclusion of a surfactant in a liquid etelcalcetide formulation reduces the formation of droplets on the internal surface of vials, effectively reducing the amount of false rejects of vials containing the liquid formulation from 15% (liquid etelcalcetide formulations without surfactant) to 0% (liquid etelcalcetide formulations with surfactant) during automated visual inspection.

### Etelcalcetide

Etelcalcetide, or “AMG 416”, or Parsabiv® refers to the compound having the chemical name: N-acetyl-D-cysteinyl-D-alanyI-D-arginyl-D-arginyl-D-arginyl-D-alanyl-D-arginamide disulfide with L-cysteine, which may be represented as:

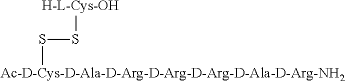

Etelcalcetide can be used in the formulations disclosed herein in salt form. Etelcalcetide, when used in the present disclosure, refers to either etelcalcetide or a salt thereof, unless otherwise indicated. Pharmaceutically acceptable salts contemplated include etelcalcetide hydrochloride, etelcalcetide hydrobromide, etelcalcetide sulfate, etelcalcetide bisulfate, etelcalcetide phosphate, etelcalcetide nitrate, etelcalcetide acetate, etelcalcetide valerate, etelcalcetide stearate, etelcalcetide benzoate, and etelcalcetide tosylate. In some embodiments, the etelcalcetide is etelcalcetide hydrochloride.

In some embodiments, the formulation comprises 0.1 mg/ml to 20 mg/mL, or 0.5 mg/ml to 15 mg/mL, or 1 mg/mL to 10 mg/mL, or 2 mg/mL to 5 mg/ml of etelcalcetide. In some embodiments, the formulation contains 1 mg/mL to 10 mg/ml of etelcalcetide. In some embodiments, the formulation contains 2 mg/ml to 5 mg/ml of etelcalcetide. In some embodiments, the formulation contains 1 mg/ml to 10 mg/ml of etelcalcetide.

In some embodiments, the formulation comprises 0.1 mg/mL, or 0.5 mg/mL, or 1 mg/mL, or 2 mg/mL, or 3 mg/mL, or 4 mg/mL, or 5 mg/mL, or 6 mg/mL, or 7 mg/mL, or 8 mg/mL, or 9 mg/mL, or 10 mg/mL, or 15 mg/mL, or 20 mg/mL, or 25 mg/mL, or 30 mg/mL, or 35 mg/mL, or 40 mg/mL, or 45 mg/mL, or 50 mg/mL, or 55 mg/mL, or 60 mg/mL, or 65 mg/mL, or 70 mg/mL, or 75 mg/mL, or 80 mg/mL, or 85 mg/mL, or 90 mg/mL, or 95 mg/mL, or 100 mg/ml of etelcalcetide.

In some embodiments, the formulation comprises 0.1 mg/mL to 20 mg/ml of etelcalcetide, based upon etelcalcetide free base weight. In some embodiments, the formulation comprises 0.1 mg/ml to 20 mg/mL, or 0.5 mg/ml to 15 mg/mL, or 1 mg/ml to 10 mg/mL, or 2 mg/mL to 5 mg/mL etelcalcetide, based upon etelcalcetide free base weight. In some embodiments, the formulation contains 1 mg/ml to 10 mg/mL etelcalcetide, based upon etelcalcetide free base weight. In some embodiments, the formulation contains 2 mg/mL to 5 mg/ml of etelcalcetide, based upon etelcalcetide free base weight.

In some embodiments, the formulation comprises 0.1 mg/mL, or 0.5 mg/mL, or 1 mg/mL, or 2 mg/mL, or 3 mg/mL, or 4 mg/mL, or 5 mg/mL, or 6 mg/mL, or 7 mg/mL, or 8 mg/mL, or 9 mg/mL, or 10 mg/mL, or 15 mg/mL, or 20 mg/mL, or 25 mg/mL, or 30 mg/mL, or 35 mg/mL, or 40 mg/mL, or 45 mg/mL, or 50 mg/mL, or 55 mg/mL, or 60 mg/mL, or 65 mg/mL, or 70 mg/mL, or 75 mg/mL, or 80 mg/mL, or 85 mg/mL, or 90 mg/mL, or 95 mg/mL, or 100 mg/mL etelcalcetide, based upon etelcalcetide free base weight.

### Buffers

The formulations described herein comprise a physiologically acceptable buffering agent that maintains the pH of the formulation in the desired range. Any buffer that is capable of maintaining the pH of the formulation at any pH or within any pH range provided above is suitable for use in the formulations of the present disclosure, provided that it does not react with other components of the formulation, cause visible precipitates to form, or otherwise cause the active ingredient to become chemically destabilized. In some embodiments, the buffer is succinate, citrate, malate, edentate, histidine, acetate, adipate, aconitate, ascorbate, benzoate, carbonate, bicarbonate, maleate, glutamate, lactate, phosphate, and tartrate, or a mixture of these buffers. In some embodiments, the buffer is a succinate buffer. In some embodiments, the buffer is sodium succinate.

The concentration of the buffer is selected so that pH stabilization as well as sufficient buffering capacity is provided. In some embodiments, the buffer is present in the formulation at a concentration of 0.5 to 100 mmol/L, 0.75 to 50 mmol/L, 1 to 20 mmol/L, or 10 to 20 mmol/L. In other embodiments, the buffer is present at 5 mmol/L, at 10 mmol/L, at 15 mmol/L or 20 mmol/L.

In some embodiments, the buffer is present in the formulation at a concentration of 0.5 mmol/L, 1 mmol/L, or 2 mmol/L, or 3 mmol/L, or 4 mmol/L, or 5 mmol/L, or 6 mmol/L, or 7 mmol/L, or 8 mmol/L, or 9 mmol/L, or 10 mmol/L, or 15 mmol/L, or 20 mmol/L, or 25 mmol/L, or 30 mmol/L, or 35 mmol/L, or 40 mmol/L, or 45 mmol/L, or 50 mmol/L, or 55 mmol/L, or 60 mmol/L, or 65 mmol/L, or 70 mmol/L, or 75 mmol/L, or 80 mmol/L, or 85 mmol/L, or 90 mmol/L, or 95 mmol/L, or 100 mmol/L.

In some embodiments, the buffer is present in the formulation at a concentration of 10 mmol/L. In some embodiments, succinate is present in the formulation at a concentration of 10 mmol/L.

In some embodiments, the formulation has a pH of about 2.0 to about 5.0, a pH of about 2.5 to about 4.5, a pH of about 2.5 to about 4.0, a pH of about 3.0 to about 3.5 or a pH of about 3.0 to about 3.6. In some embodiments, the formulation has a pH of about 2, a pH of about 2.5, a pH of about 3.0, a pH or about 3.3, a pH of about 3.5 or a pH of about 4.0. In some embodiments, the formulation has a pH of 2.0 to 5.0, a pH of 2.5 to 4.5, a pH of 2.5 to about 4.0, a pH of 3.0 to 3.5 or a pH of 3.0 to 3.6.

### Surfactants

The pharmaceutical formulations described herein comprise a surfactant. Exemplary surfactants include but are not limited to, polysorbate 20, polysorbate 40, polysorbate 60, polysorbate 80, poloxamer 188, poloxamer 407, triton X-100, polyoxyethylene, PEG 3350, PEG 4000, sodium dodecyl sulfate (SDS), polyvinyl alcohol (PVA), phosphatidylcholine (PC), Pluronic F-127, or a combination thereof.

In some embodiments, the formulation comprises a surfactant at a concentration of 0.001% to 5% w/v (or 0.001% to 0.5%, or 0.004 to 0.5% w/v or 0.001 to 0.01% w/v or 0.004 to 0.01% w/v). In some embodiments, the formulation comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some embodiments, the formulation comprises a surfactant at a concentration of 0.001% to 0.5% w/v. In some embodiments, the formulation comprises a surfactant at a concentration of 0.001 to 0.01% w/v. In some embodiments, the formulation comprises a surfactant at a concentration of 0.001%, 0.002%, 0.003%, 0.004%, 0.005%, 0.006%, 0.007%, 0.008%, 0.009%, 0.01%, 0.05%, 0.1%, 0.2%, 0.3%, 0.4%, to 0.5% w/v. In some embodiments, the formulation comprises a surfactant incorporated in a concentration of 0.001% to 0.01% w/v. In some embodiments, the surfactant is polysorbate 20 and the polysorbate 20 is present in a concentration of 0.01% w/v.

### Other Excipients

It is generally desirable for a formulation to be administered by intravenous or other parenteral route to be isotonic with bodily fluids. In some embodiments, the formulations of the disclosure contain a physiologically acceptable tonicity modifier. Exemplary tonicity modifiers include, but are not limited to, sodium chloride, mannitol, sucrose, dextrose, sorbitol, potassium chloride, and mixtures thereof. In some embodiments, the tonicity modifier is sodium chloride.

In some embodiments, the formulation comprises a tonicity modifier in an amount sufficient to make the liquid formulation approximately isotonic with bodily fluids (i.e., 270 to 300 mOsm/L) and suitable for parenteral injection into a mammal, such as a human subject, into dermal, subcutaneous, or intramuscular tissues or IV. Isotonicity can be measured by, for example, using a vapor pressure or ice-freezing type osmometer. Depending upon the concentrations of the other components in the formulation, sodium chloride is present in the formulation at a concentration of 7.0 to 10 mg/mL, 7.5 to 9.5 mg/mL, or 8.0 to 9.0 mg/mL. In a one embodiment, sodium chloride is present in the formulation at a concentration of 8.5 mg/mL. In other embodiments, sodium chloride is present in the formulation at a concentration of 7.0 to 10 mg/ml, 7.5 to 9.5 mg/mL, or 8.0 to 9.0 mg/mL.

The formulations of the disclosure may include other conventional pharmaceutical carriers, excipients or adjuvants. For example, the formulations of the present invention may include stabilizing agents (e.g., EDTA and/or sodium thiosulfate) or preservatives (e.g., benzyl alcohol).

In some embodiments, the pharmaceutical formulation comprises 5 mg/ml of etelcalcetide hydrochloride in aqueous solution, based upon etelcalcetide free base weight, 10 mmol/L succinate buffer, 0.01% (w/v) polysorbate 20, at pH 3.2.

### Therapeutic Use of the Formulations

The formulations described herein are useful for the treatment or amelioration of hyperparathyroidism, hypercalcemia and/or bone disease in a subject in need thereof. The terms “subject in need” or those “in need of treatment” includes those already with the disorder, as well as those in which the disorder is to be prevented. The “subject in need” or “patient” includes human and other mammalian subjects that receive either prophylactic or therapeutic treatment. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having hyperparathyroidism, hypercalcemia and/or bone disease, by the administration of a formulation described herein to a subject in need thereof.

Preferably, the pharmaceutical formulation is administered parenterally, e.g., intravenously, subcutaneously, or intramuscularly. Parenteral administration may be achieved by injection, such as bolus injection, or by infusion, such as continuous infusion. Administration may be achieved via depot for long-term release. In some embodiments, the formulation is administered intravenously by an initial bolus followed by a continuous infusion to maintain therapeutic circulating levels of drug product. In some embodiments, the formulation is administered as a one-time dose. Pharmaceutical formulations may be administered using a medical device. Examples of medical devices for administering pharmaceutical formulations are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

In some embodiments, the formulations disclosed herein may be used alone or in combination with one or more other therapeutically effective agents. Such other therapeutic agents include, but are not limited to, treatment with anti-resorptive bisphosphonate agents, such as alendronate and risedronate; integrin blockers, such as αvp3 antagonists; conjugated estrogens used in hormone replacement therapy, such as PREMPRO™ PREMARIN™ and ENDOMETRION™; selective estrogen receptor modulators (SERMs), such as raloxifene, droloxifene, CP-336, 156 (Pfizer) and iasofoxifene; cathespin K inhibitors; vitamin D therapy; vitamin D analogs, such as ZEMPLAR™ (paricaicitol); CALCIJEX® (cafcitriol), HECTOROL® (doxercalciferol), ONE-ALPHA® (alfacalcidol) and the analogs in development from Cytochroma known as CTA-018, CTAP201 and CTAP101; other calcimimetics such as Sensipar® (cinacalcet); inhibitors of type II sodium-dependent phosphate transporter family, SLC34 (including the two renal isoforms NaPi-Ha and NaPi-IIc, and the intestinal NaPi-IIb transporter); phosphatonins (including FGF-23, sFRP4, MEPE or FGF-7); low dose PTH treatment (with or without estrogen); calcitonin; inhibitors of RANK ligand; antibodies against RANK ligand, osteoprotegrin; adensosine antagonists; and ATP proton pump inhibitors.

### Kits

As an additional aspect, the described herein are kits which comprise one or more pharmaceutical formulations described herein packaged in a manner which facilitates their use for administration to subjects. In one embodiment, such a kit includes a formulation described herein (e.g., a formulation comprising etelcalcetide, a buffer and a surfactant as described herein), packaged in a container such as a sealed bottle, vessel, single-use or multi-use vial, prefilled syringe, or prefilled injection device, optionally with a label affixed to the container or included in the package that describes use of the compound or formulation for treatment of a subject in need thereof. In one aspect, the formulation is packaged in a unit dosage form. The kit may further include a device suitable for administering the formulation according to a specific route of administration. Preferably, the kit contains a label that describes use of the formulation described herein.

The pharmaceutical formulations described herein can be formulated in various forms, e.g., in solid, liquid, frozen, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts.

Generally, various storage and/or dosage forms are conceivable for the pharmaceutical formulation of the invention, depending, i.e., on the intended route of administration, delivery format and desired dosage (see, for example, Remington's Pharmaceutical Sciences, 22nd edition, Oslo, A., Ed., (2012). The skilled person will be aware that such choice of a particular dosage form may for example influence the physical state, stability, rate of in vivo release and rate of in vivo clearance of an antibody.

For instance, the primary vehicle or carrier in a pharmaceutical formulation may be either aqueous or non-aqueous in nature. A suitable vehicle or carrier may be water for injection, physiological saline solution or artificial cerebrospinal fluid, possibly supplemented with other materials common in formulations for parenteral administration. Neutral buffered saline or saline mixed with serum albumin are further exemplary vehicles.

### EXAMPLES

### Example 1—Surfactant Addition Reduced Droplet Formation in Etelcalcetide Formulations

A liquid etelcalcetide formulation was assessed for viscosity and surface tension. Type 1B vials (3cc) were washed with room temperature Milli Q Water and depyrogenated for 2 hours at 260° C. A liquid etelcalcetide formulation (5 mg/ml etelcalcetide, 10 mM succinic acid, 0.85% (w/v) sodium chloride in WFI pH 3.25) was prepared and aliquoted into 10 mL volumes. It was determined that the formulation had low viscosity (1.013 cP) and high surface tension (72.690 mN/m), which increases the propensity of droplet formation on the inner surface of the formulation vials.

Stock solutions of surfactants of 10% polysorbate 20 (PS20), 0.5% polyvinyl alcohol (PVA), 1% sodium dodecyl sulfate (SDS), 0.01% phosphatidylcholine (PC), 1% Pluronic F-127, and 1% PEG 4000 were prepared. The quantum satis of the stock solutions were dispensed into 10 mL aliquots to produce drug products with 0.005% PS20, 0.01% PS20, 0.05% PS20, 0.01% PVA, 0.01% SDS, 0.0001% PC, 0.01% Pluronic F-127, and 0.01% PEG4000, respectively.

The commercial fill volume (0.6 mL) of the etelcalcetide formulation with each surfactant addition was dispensed manually into 3 replicates per formulation and surfactant concentration sample. The vials were then capped with a 13 mm stopper and sealed. Finally, the representative samples of each drug product were isolated for a side-to-side visual comparison.

Furthermore, an additional 250 samples of 0.05%, 0.01%, and 0.005% PS20 were produced in Amgen Manufacturing Limited for false eject evaluation at the commercial line.

The vials were assessed via automated visual inspection. The percentage of false rejects was determined and is shown in the table below.

| Concentration of PS20 (w/v) | Percentage of False Rejects |
|---|---|
| 0% | 2.25% |
| 0.005% | 0.17% |
| 0.01% | 0.0% |
| 0.05% | 0.5% |

Commercial process monitoring results showed that the control etelcalcetide formulations (lacking the surfactant) had about 15% false rejects via automated visual inspection. In contrast, results showed that the addition of 0.01% PS20 to the etelcalcetide formulation resulted in zero false rejects via automated visual inspection. Based on knowledge of formulation performance due to high surface tension and low viscosity, and the effects of surfactants on surface tension, it is expected that the use of surfactants other than PS20 (e.g., polysorbate 40, polysorbate 60, polysorbate 80, poloxamer 188, poloxamer 407, triton X-100, polyoxyethylene, PEG 3350, PEG 4000, sodium dodecyl sulfate (SDS), polyvinyl alcohol (PVA), phosphatidylcholine (PC) or Pluronic F-127) in etelcalcetide formulations would also result in reduced false rejects compared to etelcalcetide formulations lacking the surfactant.

What is claimed is:

1. A pharmaceutical formulation comprising etelcalcetide or salt thereof and a surfactant in aqueous solution, wherein the formulation has a pH of 2 to 5.

2. The formulation of claim 1, wherein the formulation has a pH of 3 to 4.

3. The formulation of claim 1 or claim 2, wherein the pH is maintained by a pharmaceutically acceptable buffer.

4. The formulation of claim 3, wherein the buffer comprises succinate, citrate, malate, edentate, histidine, acetate, adipate, aconitate, ascorbate, benzoate, carbonate, bicarbonate, maleate, glutamate, lactate, phosphate, and tartrate or a mixture thereof.

5. The formulation of claim 3 or claim 4, wherein the buffer comprises succinate.

6. The formulation of any one of claims 1-5, wherein the surfactant comprises polysorbate 20, polysorbate 40, polysorbate 60, polysorbate 80, poloxamer 188, poloxamer 407, triton X-100, polyoxyethylene, PEG 3350, PEG 4000, sodium dodecyl sulfate (SDS), polyvinyl alcohol (PVA), phosphatidylcholine (PC), Pluronic F-127, or a mixture thereof.

7. The formulation of claim 6, wherein the surfactant comprises polysorbate 20.

8. The formulation of any one of claims 1-7, wherein the surfactant is present in an amount of 0.005% (w/v) to 0.01% (w/v).

9. The formulation of any one of claims 1-8, wherein the etelcalcetide or salt thereof is present in the formulation at a concentration of 1 mg/mL to 20 mg/mL, based upon etelcalcetide free base weight.

10. The formulation of any one of claims 1-9, wherein the etelcalcetide or salt thereof is present in the formulation at a concentration of 2.5 mg/mL to 10 mg/mL, based upon etelcalcetide free base weight.

11. The formulation of any one of claims 1-10, further comprising a tonicity modifier.

12. The formulation of claim 11, wherein the tonicity modifier comprises sodium chloride, mannitol, sucrose, dextrose, sorbitol, potassium chloride, or mixtures thereof.

13. The formulation of claim 11, wherein the tonicity modifier comprises sodium chloride.

14. A pharmaceutical formulation comprising
etelcalcetide or salt thereof at a concentration of 1 mg/ml to 20 mg/ml in aqueous solution, based upon etelcalcetide free base weight;
a succinate buffer at a concentration that maintains the formulation at a pH of about 3 to 4;
a surfactant, and
sodium chloride at a concentration such that the formulation is approximately isotonic.

15. The formulation of claim 14, wherein the surfactant comprises polysorbate 20, polysorbate 40, polysorbate 60, polysorbate 80, poloxamer 188, poloxamer 407, triton X-100, polyoxyethylene, PEG 3350, PEG 4000, sodium dodecyl sulfate (SDS), polyvinyl alcohol (PVA), phosphatidylcholine (PC), Pluronic F-127, or a mixture thereof.

16. The formulation of claim 14 or claim 15, wherein the surfactant comprises PS20.

17. The formulation of any one of claims 14-16, wherein the surfactant is present in an amount of 0.005% (w/v) to 0.05% (w/v).

18. A pharmaceutical formulation comprising etelcalcetide hydrochloride and a surfactant in aqueous solution, wherein the formulation has a pH of 2 to 5.

19. The formulation of claim 18, wherein the formulation has a pH of 3 to 3.5.

20. The formulation of claim 18 or claim 19, wherein the pH is maintained by a pharmaceutically acceptable buffer.

21. The formulation of claim 20, wherein the buffer comprises succinate, citrate, malate, edentate, histidine, acetate, adipate, aconitate, ascorbate, benzoate, carbonate, bicarbonate, maleate, glutamate, lactate, phosphate, and tartrate or a mixture thereof.

22. The formulation of claim 20 or claim 21, wherein the buffer comprises succinate.

23. The formulation of any one of claims 18-22, wherein the surfactant comprises polysorbate 20, polysorbate 40, polysorbate 60, polysorbate 80, poloxamer 188, poloxamer 407, triton X-100, polyoxyethylene, PEG 3350, PEG 4000, sodium dodecyl sulfate (SDS), polyvinyl alcohol (PVA), phosphatidylcholine (PC), Pluronic F-127, or a mixture thereof.

24. The formulation of any one of claims 18-23, wherein the surfactant comprises polysorbate 20.

25. The formulation of any one of claims 18-24, wherein the surfactant is present in an amount of 0.005% (w/v) to 0.05% (w/v).

26. The formulation of any one of claims 18-25, wherein the etelcalcetide hydrochloride is present in the formulation at a concentration of 1 mg/mL to 15 mg/mL, based upon etelcalcetide free base weight.

27. The formulation of any one of claims 18-26, wherein the etelcalcetide hydrochloride is present in the formulation at a concentration of 2.5 mg/mL to 10 mg/mL, based upon etelcalcetide free base weight.

28. The formulation of any one of claims 18-27, further comprising a tonicity modifier.

29. The formulation of claim 28, wherein the tonicity modifier comprises sodium chloride, mannitol, sucrose, dextrose, sorbitol, potassium chloride, or mixtures thereof.

30. The formulation of claim 29, wherein the tonicity modifier comprises sodium chloride.

31. A pharmaceutical formulation comprising 1 mg/mL to 20 mg/ml of etelcalcetide hydrochloride in aqueous solution, based upon etelcalcetide free base weight, a succinate buffer that maintains the formulation at a pH of about 3 to 4, a surfactant, and a concentration of sodium chloride wherein the formulation is approximately isotonic.
